SEQUENCE LISTING (4 sequences)

SEQ ID NO: 1 - protein, 144 aa, Mycobacterium tuberculosis
  MISC_FEATURE: Protein of Mycobacterium tuberculosis H37Rv, (NCBI No. CAA17245)
MATTLPVQRHPRSLFPEFSELFAAFPSFAGLRPTFDTRLMRLEDEMKEGRYEVRAELPGVDPDKDVDIMVRDGQLTIKAERTEQKDFDGRSEFAYGSFVRTVSLPVGADEDDIKATYDKGILTVSVAVSEGKPTEKHIQIRSTN

SEQ ID NO: 2 - protein, 70 aa, Mycobacterium tuberculosis
  MISC_FEATURE: ESAT-6 Protein of Mycobacterium tuberculosis H37Rv, (NCBI No AAC83446)
MTEQQWNFAGIEAAASAIQGNVTSIHSLLDEGKQSLTKLAAAWGGSGSEAYQGVQQKWDATATELNNALQ

SEQ ID NO: 3 - protein, 100 aa, Mycobacterium tuberculosis
  MISC_FEATURE: CFP-10 Protein of Mycobacterium tuberculosis H37Rv, (NCBI No CAA17966.1)
MAEMKTDAATLAQEAGNFERISGDLKTQIDQVESTAGSLQGQWRGAAGTAAQAAVVRFQEAANKQKQELDEISTNIRQAGVQYSRADEEQQQALSSQMGF

SEQ ID NO: 4 - protein, 325 aa, Mycobacterium tuberculosis
  MISC_FEATURE: 85B Protein of Mycobacterium tuberculosis H37Rv, (NCBI No NP_216402)
MTDVSRKIRAWGRRLMIGTAAAVVLPGLVGLAGGAATAGAFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

(19) United States
(12) Patent Application Publication
(10) Pub. No.: US 2010/0093007 A1
(43) Pub. Date: Apr. 15, 2010

(54) Method of Diagnosis of Tuberculosis Related Immune Restoration Syndrome (IRS)

(75) Inventors: Brigitte Autran, Paris (FR); Anne Bourgarit, Paris (FR); Guislaine Carcelain, Issy Les Moulineaux (FR); Valérie Martinez, Paris (FR); Brigitte Gicquel, Paris (FR); Daniel Sereni, Paris (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Institut National de la Sante et de la Recherche Medicale (INSERM), Paris (FR)

(21) Appl. No.: 11/992,562

(22) PCT Filed: Sep. 7, 2006
(86) PCT No.: PCT/EP2006/066152

§ 371 Date: Mar. 24, 2009

(30) Foreign Application Priority Data
Sep. 27, 2005 (EP) 05291998.2

(51) Int. Cl. G01N 33/566

(52) U.S. Cl. 435/7.94; 435/7.92

(57) ABSTRACT

The present invention relates to a method and kit of diagnosis of Immune Restoration Syndrome associated with tuberculosis (TB-IRS) in patients infected with tuberculosis (TB) as well as in HIV co-infected patients comprising detecting an acute increase in Th1 response following exposure to mycobacterial extract, referred as tuberculin or PPD (Purified Protein Derivative) as well as to the 16 kDa protein, but not to ESAT-6 or CFP-10, two antigens from mycobacterium tuberculosis.

The present invention relates to a method and kit of diagnosis of Immune Restoration Syndrome associated with tuberculosis (TB-IRS) in patients infected with tuberculosis (TB) as well as in HIV co-infected patients comprising detecting an acute Th1 response following exposure to mycobacterial extract, referred as tuberculin or PPD (Purified Protein Derivative) and/or to the 16 kDa protein, but not to ESAT-6 or CFP-10 antigens from mycobacterium tuberculosis.

### BACKGROUND OF THE INVENTION

First described in HIV-infected patients with Mycobacterium avium complex infection receiving zidovudine monotherapy [1], Immune Restoration Syndrome (IRS) in patients suffering from tuberculosis became extremely frequent (29-36%) [2] with the Highly Active Antiretroviral Therapy (HAART) era. IRS can also occur after initiation of TB therapy in HIV-negative patients, though at a lower frequency.

Classically, IRS occurs in patients co-infected with tuberculosis (TB) and HIV, naïve of HAART, with advanced HIV disease, when both therapies are initiated simultaneously or at a short interval. Usually, clinical symptoms include fever, lymphadenopathy, worsening of respiratory and other initial TB symptoms [2]. Definition IRS major criteria have been proposed [3] whatever the underlying opportunistic event including an atypical presentation of opportunistic infection (OI) or tumours in patients responding to HAART (decrease in plasma HIV RNA level more than 1 log₁₀ copies/ml), without any alternative explanation (drug resistance, other OI) and with evidence for immune restoration, as minor criteria.

However, IRS raises three clinically relevant issues: first, diagnosis is difficult as IRS should be distinguished from treatment failure or resistance or with other opportunistic diseases; second, IRS treatment in some life-threatening cases is still empirically based on steroids, which are uneasy to introduce in such immunocompromised patients; third, determination of IRS risk factors could help clinicians to determine their therapeutic strategy, i.e. delaying or not HAART introduction [4].

Therefore, there is still as of today a strong need for a reliable test for diagnosis of IRS.

The pathophysiology of IRS is not yet totally elucidated. Acute proinflammatory cytokine (IL-6) production has been demonstrated during IRS [5, 6] but its source remains unknown. In addition, a strong and dominant hypothesis is that IRS would be associated to acute restoration of mycobacteria-specific T-lymphocyte response with either recirculation or proliferation of memory T-cells after HAART has reduced the plasma viral load (VL) [7, 8]. This hypothesis is only suggested but not yet demonstrated, except for the restoration of DTH skin test to PPD [9]. Similarly in HIV-negative TB-infected patients, it is hypothesized that IRS reflects exacerbation of TB-specific Th1 immunity.

In connection with the invention, we conducted a prospective multicenter study of patients co-infected with HIV and TB. We found that Mycobacteria-specific (PPD) Th1 IFN-γ-producing cells increased sharply during IRS, while the CMV-, HIV p24-specific responses tested as controls did not. In addition, only very few IRS+ patients had ESAT-6-specific responses albeit at low levels during IRS; and IRS− patients did not develop acute PPD-specific responses. These findings correlated with a peak of PPD-specific Th1 cytokines such as IFN-γ, IL-2, IL-12, IP10 and MIG but we did not detect Th2 cytokines; other inflammatory chemokines or cytokines were also detected such as TNF-α, IL-6, IL-1β, IL-10, RANTES and MCP-1. We also identified the acute release of Th1 specific cytokines in response to stimulation with the 16 kDa protein antigen.

Thus, we provide here a new diagnostic test of TB-IRS consisting of detecting the acute exacerbation of Th1 responses against mycobacterial antigens in patients co-infected with HIV and TB as well as patients with TB alone.

### DESCRIPTION

In a first embodiment, the invention is aimed at a method of in vitro diagnosis of Immune Restoration Syndrome (IRS) in patients co-infected with mycobacterium tuberculosis (TB) as well as in patients infected with TB alone, comprising the step consisting of:

a) incubating peripheral blood mononuclear cells (PBMC) or whole blood of said patients in a culture medium with mycobacterial antigens selected from mycobacterial extract referred as PPD (purified protein derivative), and/or with the 16 kDa protein (SEQ ID No. 1),
b) incubating peripheral blood mononuclear cells (PBMC) or whole blood of said patients in a culture medium with at least one antigen from mycobacterium tuberculosis selected from ESAT-6 (SEQ ID No. 2), CFP-10 (SEQ ID No. 3) and 85B (SEQ ID No. 4) as negative control,
c) detecting the level of the Th1 response against said mycobacterial antigens described in a) compared to said mycobacterial antigens in b);
wherein an increase above 250% of Th1 response, or a strong reactivity in the Th1 response measured at one time point against one of the antigens as defined in a) compared to the antigens as defined in b) or compared to non-stimulated cells or control cells stimulated with non-mycobacterial antigens is indicative of IRS.

In a preferred embodiment, step a) and step b) are simultaneous.

In another preferred embodiment of the method of the present invention, the increase of Th1 response, or the reactivity measured at one time point, against one of the said antigens as defined in step b) compared to non-stimulated cells or control cells stimulated with non-mycobacterial antigens is null or minimal.

This test is based on our discovery that in TB-IRS, there is an acute increase of Th1 response specifically for PPD and for the 16 kDa protein but not for other mycobacterial antigens such as ESAT-6, CFP-10 and 85B. By acute increase, it will be understood herein an increase typically above 250% or even comprised between 500% and 5000% above background or at least above 250% (for example above 300%, 400% or even 500%) of Th1 response compared to control cells. By “no or minimal reactivity”, it will be understood insignificant increase of the Th1 response compared to control cells, for example below 200% and typically below 100% or 50%.

The above increase in % can be calculated by the skilled in the art by simply dividing the quantitative measure obtained in Th1 tests (for example cytokine release tests such as in an IFN-gamma release test) obtained for the antigens as defined in step a) with the quantitative measure obtained for the antigens as defined in step b) or obtained with non-stimulated cells or control cells stimulated with non-mycobacterial antigens. ESAT-6, 85B and CFP-10 are antigens isolated from mycobacterium tuberculosis (Mtb) while PPD is composed of extracts from both Mtb and BCG (the Bacille of Calmette et Guérin used as a universal vaccine against Mtb, are well known in the art and can be obtained from several commercial sources including the PPD marketed by Statens Serum Institute, Copenhagen, Denmark. PPD or tuberculin is obtained by extracting proteins from a mixture of Mtb and BCG and is commonly employed in testing for the presence of cellular immunity or Th1 response generated either against BCG or against Mtb. For example, it can be obtained from the Tubersol® of Connaught Laboratories Limited prepared from a large Master Batch, Connaught Tuberculin (CT68). The ESAT-6 protein is a major secreted antigen which has been purified from M. tuberculosis short-term culture filtrates as described in Harboe et al., 1996; Sorensen et al., 1995). As referred herein ESAT-6, CFP-10 and 85B can be obtained from cell lysate and purification or by recombinant technique. For example ESAT6 can be obtained as a recombinant protein from Statens Serum Institute.

Tuberculin or PPD (purified protein derivative) from mycobacterial extracts differs from ESAT-6 (early secreted antigenic target 6) CFP-10 (culture filtrate protein 10), and 85B which are encoded by genes located only within the M. tuberculosis genome and are not contained in BCG, because PPD is also containing other antigens that are shared with BCG substrains and with several nontuberculous mycobacterial species with low or no pathogenicity.

Sequences of these antigens are also displayed in the Sequence listing incorporated herein.

When a sequential measurement in Th1 responses is not feasible the test is based on a single blood sample harvested at a single time point during or immediately after the IRS and comprises performing a quantification of antigen-specific T-cells by ELISPOT or any assay detecting a Th1 response, such as a quantification of mycobacterial-specific Th1 cells producing IFN-γ to one of the antigens as defined in step a) but not to the antigens as defined in step b).

Here, a strong reactivity is defined by a result above 500 SFC (ELISspot-forming-cells)/10⁶ PBMC, for example a SFC above 500, 1000, or 2000 or higher against the antigens as defined in step a), after subtraction of the mean background obtained with cells alone, is an indication of IRS; while simultaneously no or minimal SFC are detected against ESAT-6, 85B, CFP-10, such as SFC typically comprised between 500 and 0/10⁶ PBMC. Alternatively a 250% or more, higher Th1 response against the antigens as defined in step a) compared to the antigens as defined in step b) is an indication of IRS.

Preferably, the mean background obtained with cells alone is egal to or less than 100 SFC/10⁶ PBMC.

As a result, the percentage SFC antigen of a)/SFC no antigen gives the threshold indicative of TB-IRS, i-e above 250%, more specifically above 500%.

As a result, the percentage SFC antigen of a)/SFC antigen of b) gives the threshold indicative of TB-IRS, i-e above 250%.

The ELISPOT method can be replaced by any method that detects a Th1 response, i.e. measuring T cells producing other Th1 cytokines by ELISpot or quantification of Th1 cytokines released by T cells either an intra-cellular cytokine staining method combined to flow cytometry or fluorescent microscopic examination and quantification.

Alternatively or concurrently the test may comprise a method collecting culture supernatants obtained in step a) and b), quantifying at least one Th1 specific cytokine or IL-2, IL-2R, IL-12p40, IL-15, IL-17, IFN-γ, TNF-α, GMCSF, MIP-1α, MIP-1β, MCP-1, MIG, RANTES, IP-10 in the culture supernatants of cells stimulated by the antigens as defined in step a) and b) compared to unstimulated cells.

Such analysis can be performed using a multiplexed sandwich immunoassay or an individual cytokine-specific sandwich immuno-assay.

In one particular aspect of these alternative or concurrent test of the present invention, the quantification of at least one Th1 specific cytokine comprises quantification of IFN-γ production by ELISA or RIA.

Preferably, peripheral blood mononuclear cells in step a) are incubated from about 6 to 72 hours. In addition, peripheral blood mononuclear cells in step a) are advantageously incubated with said “step a)” antigens PPD and/or the 16 kDa protein at about 1 μg/ml and separately with said “step b)” antigen ESAT-6 recombinant protein at about 1 μg/ml. PBMC may also be incubated with CFP-10 or 85B as negative control.

Control cells are selected from cells incubated with no antigen or non-mycobacterium antigen. For example, it is possible to use CMV extracts, HIV-1 p24, phytohaemagglutinin. Culture with medium alone serves as negative control to measure background.

The method of the invention is performed in the course of TB, and especially in the course of Highly Active Antiretroviral Therapy (HAART) in HIV positive patients co-infected with tuberculosis. More specifically, it is performed in patients receiving simultaneous or sequential HAART and tuberculosis treatment.

HAART is well known in the art and includes several protocols of combined treatment with Nucleoside Analog Reverse Transcriptase Inhibitors (NARTIs) or (NRTIs), NonNucleoside Reverse Transcriptase Inhibitors (NNRTIs), and Protease inhibitors (PIs) or other molecules inhibiting HIV entry into target cells or inhibiting virus DNA integration or any other new antiretroviral molecule capable to block significantly HIV replication and to allow CD4 T cell reconstitution.

In a second embodiment, the invention is directed to a kit for performing the method as defined above.

Thus, the invention relates to a diagnostic kit of Immune Restoration Syndrome (IRS) in TB patients or patients co-infected with tuberculosis (TB) and HIV, comprising mycobacterial antigens selected from mycobacterial extract referred as PPD (purified protein derivative) or/and the 16 kDa protein (SEQ ID No. 1), and at least one antigen from mycobacterium tuberculosis associated with Mtb virulence selected from ESAT-6 (SEQ ID No. 2), CFP-10 (SEQ ID No. 2) and 85B (SEQ ID No. 3). For example, the kit comprises PPD and ESAT-6 or PPD, ESAT-6 and CFP-10 and/or 85B. In another example, the kit comprises the 16 kDa protein and at least one of ESAT-6 or CFP-10 and 85B.

In addition, the kit may comprise at least one anti-cytokine/chemokine antibody specific to the Th1 immune response bound to a solid support, such as anti-IFN-γ antibodies. The solid supports of the kit can be an ELISPOT plate and/or an ELISA plate.

In a third embodiment, the invention is directed the use of the method and kit defined above for detecting TB related Immune Restoration Syndrome (TB-IRS) in patients with TB or in patients co-infected with tuberculosis (TB) and HIV in course of HAART and tuberculosis treatment.

### FIGURES LEGEND

FIGS. 1A-1D: PPD-specific reactivity between patients with and without IRS.

The left column represents data for IRS+ patients, and the right column data for IRS−patients.

FIG. 1A: Overall results of ELISpot IFN-γ assays expressed as Spot Forming Cells (SFC)/million PBMC performed in all IRS+ and IRS− patients after 40 hours PBMC stimulation with PPD (red) or CMV (cyan).

FIGS. 1B, 1C, 1D: Representative results from one IRS+ and one IRS− patient

FIG. 1B: IFN-γ ELISpot after stimulation with PPD, ESAT6 (red) versus control antigens (CMV, HIV₁, p24) (cyan); FIGS. 1C, 1D: Luminex-measured production of: FIG. 1C: IFN-γ (▪), IL-2 [symbol] IP-10 (▴) and FIG. 1D: TNF-α [symbol] IL-6 (▪), IL-1β(▴), after in vitro PBMC stimulation with PPD (red), CMV (cyan) and medium alone in two representative IRS+ and IRS− patients.

FIG. 2: Regression curves of ELISpot quantification of PPD- and native 16 kDa-(left), ESAT-6 (right) specific cells (SFC/10⁶ PBMC) (n=37).

FIG. 3: kinetic of number of antigen-specific cells in one IRS+ patient measured by ELISpot, expressed as Spot Forming Cells (SFC)/10⁶ PBMC. T_{TB}=anti mycobacterial therapy onset, T_{IRS}=IRS time, M₃=Month 3 after HAART onset.

### EXAMPLE 1

### IRS is Induced by Mycobacterial-Specific Th1 Cells in HIV-Tuberculosis Co-Infected Patients

### 1—Patients and Methods

### 1.1.1 Patients

Twenty-nine consecutive TB-HIV co-infected untreated patients were prospectively included when beginning anti-TB treatment. Inclusion criteria were: HIV-1 infection, no previous HAART, CD4 count below 200 cells/mm³, anti-TB therapy initiated within a maximum of one week before inclusion and indication to further HAART initiation. Inclusion was secondly confirmed when M tuberculosis infection was proved (positive culture or histological findings). Patients were evaluated when initiating antimycobacterial therapy (T_{BK}) and HAART (M₀) and at 1, 3, 6 and 12 months after HAART initiation (M_{1,3,6,12}). In addition, patients with IRS (IRS+) were evaluated at IRS time (T_{IRS}) and 20 days later. IRS was defined as follows [3]: recurrence of inflammatory reaction (fever, elevated CRP), enlargement of pre-existing lesions, or development of new lesions (lymph nodes, pleuritis) with no mycobacterium resistance, no positive culture specimen, no other diagnosis and with a response to HAART (HIV-RNA decrease>1 log copies/ml). Patients who did not experience IRS within three months after HAART initiation were defined as IRS−.

The study was accepted by the Saint-Louis Hospital Institutional-Ethical-Committee and all patients signed an informed consent.

### 1.1.2 Methods

### ELISPOT Assay for Quantification of Mycobacterial-Specific Th1 Cells

Antigen-specific Th1 cells producing IFN-γ were prospectively quantified on fresh peripheral blood mononuclear cells (PBMC) by ELISpot as described [10], after a 40-hour stimulation with mycobacterial extracts (PPD, 1 μg/ml; Statens Serum Institute, Copenhagen, Denmark) and ESAT-6 recombinant protein (1 μg/ml, kindly provided by Statens Serum Institute, Copenhagen, Denmark). Controls included CMV extracts (Behring), HIV-1 p24 (Protein-Sciences), phytohaemagglutinin (Murex) and medium alone. Spots were counted using an ELISpot reader (Zeiss) and data were expressed as spot-forming-cells (SFC)/10⁶ PBMCs. Results were considered as positive if above a 50 SFC/10⁶ PBMC after subtraction of the mean background obtained with cells alone.

### Cytokine/Chemokine Production Quantification

Fresh PBMC were stimulated for cytokine/chemokine production with the same antigens as above. Culture supernatants were collected at day 2 of incubation and were cryopreserved. Quantification of 25 inflammatory and immunomodulatory cytokines/chemokines (IL-1β, IL-1RA, IL-2, IL-2R, IL-4, IL-5, IL-6, IL-7, IL-8, IL-10, IL-12p40, IL-13, IL-15, IL-17, IFN-γ, IFN-γ, TNF-α, GMCSF, MIP-1α, MIP-1β, MCP-1, MIG, RANTES, IP-10, Eotaxin) was performed for four patients (3 IRS+ and 1 IRS−) using the multiplexed sandwich immunoassay (Human 25-plex®, Biosource) based on flowmetric Luminex® technology (Luminex 100™, Clinisciences) as published [11]. The IFN-γproduction was quantified on the same supernatants by a Biosource ELISA kit.

### Lymphocyte Phenotyping

CD4 and CD8 T-lymphocyte phenotype was analysed in fresh whole blood by four-colour flow cytometry using standard methods [7] with the following antibodies: anti-HLA-DR-FITC (Immunotech), anti-CD25-PE (Becton Dickinson), anti-CD4-PerCPCy5 (Becton Dickinson) or anti-CD8-PerCP (Becton Dickinson) and anti-CD3-APC (Becton Dickinson). Stained cells were analysed on a FACScalibur (Becton Dickinson) as described.

### Statistic Analysis

Comparisons between groups were made using χ² and Student t tests as appropriate.

### 1.2 Results

Twenty-nine consecutive TB-HIV co-infected untreated patients were prospectively included when they began anti-TB treatment. Ten were then excluded for the following reasons: four had unproven tuberculosis, five moved and one was rapidly transferred to the ICU. The other 19 were followed for at least three months after initiating HAART. Seven of them (39%) experienced IRS within a median of 23 (12-85) days after M₀ IRS clinical manifestations are listed in Table 1.

TABLE 1

| | | | | | | Time to | | | |
|---|---|---|---|---|---|---|---|---|---|
| | Age | | TB | CD4/ | VL | HAART | | ΔCD4 | IRS Clinical |
| Patient | yr | sex | localisation | mm³ | log/ml | dy | HAART | M₃-M₀ | manifestations |
| 1 | 30 | F | pleuritis | 26 | 4.7 | 36 | 3TC TFV | 86 | Peritonitis, |
| | | | | | | | EFV | | tubal |
| | | | | | | | | | granuloma |
| 2 | 41 | M | liver, lymph | 9 | 5.7 | 15 | AZT 3TC | 125 | Fever, hepatitis |
| | | | nodes | | | | T20 | | |
| 3 | 43 | M | lung | 16 | 5.04 | 14 | AZT 3TC | 15 | Pericarditis |
| | | | | | | | EFV | | |
| 4 | 32 | F | lung, bone | 15 | 6.5 | 63 | 3TC D4T | 107 | Fever, |
| | | | marrow, | | | | NFV | | abdominal |
| | | | lymph nodes, | | | | | | lymph node |
| | | | liver | | | | | | swelling |
| 5 | 35 | M | lung, lymph | 115 | 5.8 | 40 | AZT 3TC | 94 | Fever, |
| | | | nodes | | | | EFV | | peritonitis, |
| | | | | | | | | | abdominal |
| | | | | | | | | | lymph node |
| | | | | | | | | | swelling |
| 6 | 32 | F | lung | 24 | 5.7 | 27 | FTC TFV | 367 | Fever, alveolar |
| | | | | | | | EFV | | pneumonitis, |
| | | | | | | | | | lymph node |
| | | | | | | | | | swelling |
| 7 | 49 | M | miliary, | 6 | 5 | 10 | TFV FTC | 14* | Fever, |
| | | | perihepatic | | | | LPV/RTV | | abdominal |
| | | | lymph nodes | | | | | | pain, lymph |
| | | | | | | | | | node swelling |
| A | 36 | M | lymph nodes | 15 | 4.96 | 39 | AZT 3TC | 71 | None |
| | | | | | | | ABC | | |
| B | 38 | M | lung, lymph | 8 | 5.6 | 20 | AZT 3TC | 53 | None |
| | | | nodes, spleen | | | | NFV | | |
| C | 38 | M | liver, lymph | 22 | 5.7 | 27 | AZT 3TC | 20 | None |
| | | | nodes, lung | | | | EFV | | |
| D | 37 | M | lung | 32 | 5.4 | 14 | AZT 3TC | 49 | None |
| | | | | | | | EFV | | |
| E | 30 | F | lung, liver, | 43 | 4.8 | 61 | AZT 3TC | −7 | None |
| | | | spleen | | | | ABC | | |
| F | 26 | F | abdominal | 47 | 5.0 | 76 | AZT 3TC | 47 | None |
| | | | lymph nodes, | | | | LPV/rtv | | |
| | | | iliitis | | | | | | |
| G | 34 | M | abdominal | 131 | 5.1 | 50 | TFV 3TC | 80 | None |
| | | | lymph nodes | | | | RTV ATZ | | |
| H | 31 | M | lung | 198 | 5.9 | 24 | AZT 3TC | −88 | None |
| | | | | | | | EFV | | |
| I | 61 | M | lung, lymph | 166 | 5.5 | 111 | 3TC DDI | 18 | None |
| | | | node | | | | EFV | | |
| J | 52 | M | lung, liver, | 219 | 4.7 | 53 | 3TC ABC | 80 | None |
| | | | spleen | | | | TFV DDI | | |
| K | 35 | M | lung | 267 | 5.2 | 23 | AZT 3TC | 74 | None |
| | | | | | | | ABC | | |
| L | 63 | M | lymph nodes, | 60 | 5.7 | 89 | AZT 3TC | 354 | None |
| | | | lung, | | | | EFV | | |
| | | | genitourinary | | | | | | |

Three patients received oral steroids therapy with rapid recovery; at time of IRS, HAART was transiently stopped for one of them. Patients with (IRS+) and without (IRS−) IRS did not differ for ethnicity, age, sex, dissemination of tuberculosis or time between T_{BK} and M₀ (27 vs. 45 days respectively, NS). Median CD4 counts at M₀ were lower, though not significantly, in IRS+ patients (median 32/mm³ for IRS+ vs. 60/mm³ for IRS−, p=0.053) and rose at M₃ by 107/mm³ in IRS+ versus 51/mm³ in IRS− patients (NS). Time to reach an undetectable viral load did not differ either.

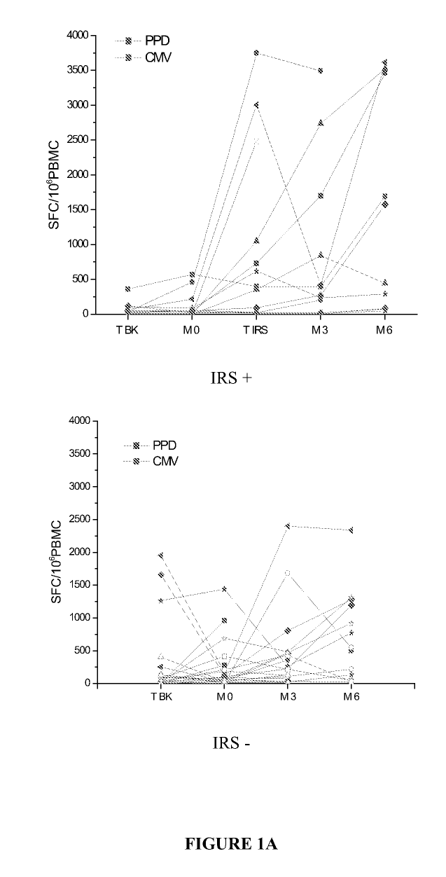

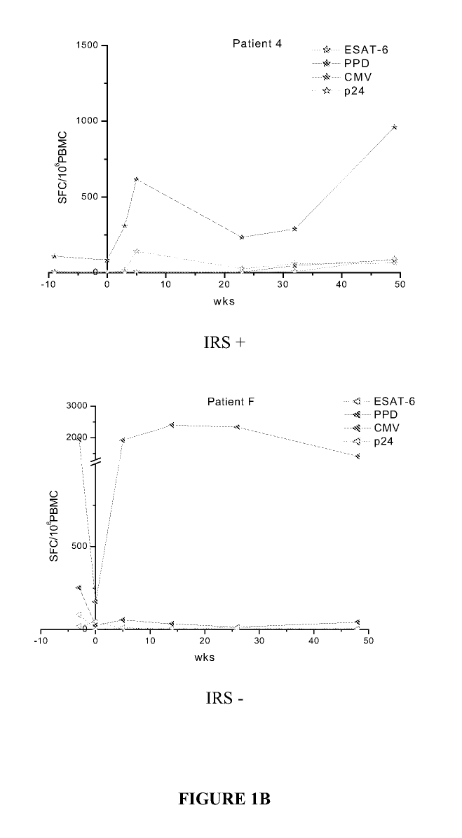

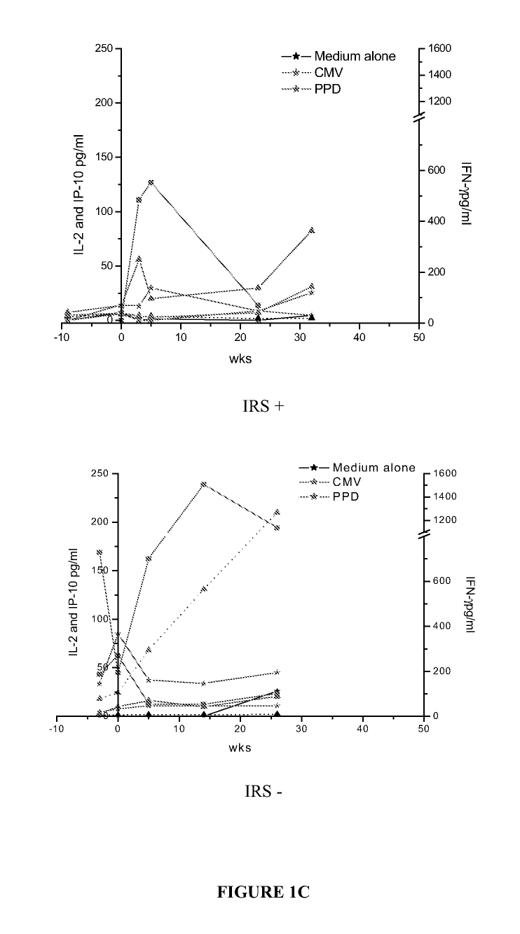

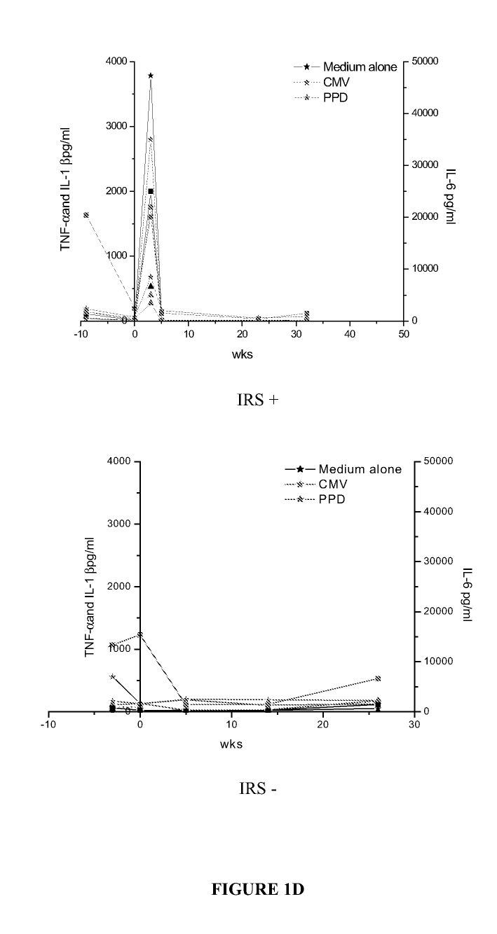

The number of PPD-specific cells producing IFN-γ did not differ at baseline (M₀) between the two groups but their proportion increased sharply during IRS and reached a median of 2970 SFC/10⁶PBMC (range 616-3744) in the 7 IRS+ patients (FIGS. 1A and B left panel). This explosive reactivity was restricted to this antigen and did not affect the CMV-specific responses in IRS+ patients. The IRS− patients did not develop acute PPD-specific responses (median 430 SFC/10⁶PBMC), except one patient who had an acute increase above 1500 SFC/10⁶PBMC at M₃ without clinical IRS; HAART at this time was no longer effective because of poor adherence. In addition, IRS− patients responded more to CMV than IRS+ patients (median 415 SFC/10⁶PBMC vs. 150 SFC/10⁶PBMC) (FIGS. 1A and 1B).

When comparing the reactivity against the two mycobacteria antigens, we found that, contrasting with PPD reactivity, only 3 IRS+ patients had ESAT-6-specific positive responses during IRS with frequencies of 58, 142 and 318 SFC/10⁶ PBMC (FIG. 1B). Furthermore, only two patients without IRS, both with CD4>100/mm³, recognized the ESAT-6 antigen during follow-up (data not shown).

An extensive proteomic analysis was also performed for four patients (3 IRS+, 1 IRS−), to evaluate the in-vitro production of 25 inflammatory and immunomodulatory cytokines/chemokines by chemiluminescence in antigen-stimulated PBMC-supernatants. Two distinct patterns were observed during IRS. First, PPD-specific IFN-γ production peaked with that of other PPD-specific Th-1 related cytokines/chemokines (IL-2, IL-12, IP10 and MIG) (FIG. 1C) but no Th2 cytokine modulation occurred (IL-4, IL-5, IL-13, IL-15). Second, inflammatory cytokines/chemokines (TNF-α, IL-6, IL-1β, IL-10, RANTES and MCP-1) (FIG. 1D) peaked, produced by both unstimulated and antigen-stimulated cells. In addition, PPD-specific cytokine/chemokine production far exceeded the CMV-specific response in IRS+ patients, while the CMV-reactivity predominated in the IRS− patient (FIG. 1D right).

Finally, whole-blood four-color flow cytometry analysis of CD3+ CD4+ and CD8+ T-cells showed that expression of activation markers (HLA-DR, CD25+) increased sharply during IRS, following exactly the same kinetics than the PPD-specific IFN-γ-producing responses (data not shown); while as expected, these markers decreased during HAART in patients without IRS.

### 1.3 Conclusion

The results of this prospective sequential study of HIV and TB co-infected patients initiating anti-TB and HAART therapies provide the first demonstration that IRS do correspond to an exacerbation of TB-specific Th1 responses with proinflammatory events during the immune reconstitution allowed by HAART.

It is well known that recovery of a PPD-specific response is a characteristic of immune reconstitution with HAART [12-15]. Our prospective study differs from others by clearly showing an exceptionally acute, intense and reproducible kinetic of the PPD-specific response concurrent with the acute inflammatory clinical symptoms described as IRS. This abrupt response was absent in only one IRS+ patient who stopped HAART during IRS and received corticosteroids, while, one IRS− patient showed a similar peak though with no clinical diagnosis of IRS but with poor HAART observance and immuno-virological control. Such findings had not been demonstrated yet possibly due to the retrospective characteristics of previous studies.

In addition, we show here that the mycobacteria-specific response is limited to some persistent antigens contained in PPD. Indeed, the weak response to the mycobacterial secreted protein ESAT-6 suggests that ESAT-6 either does not persist after antimycobacterial therapy or does not stimulate the T-cells involved in IRS. This finding is critical for diagnosis because new tuberculosis-diagnostic tests based on ESAT-6 [16] would have missed this acute response.

The sharp amplification of IFN-gamma producing mycobacteria-specific T cells was associated with an acute burst of other Th1 and proinflammatory cytokines/chemokines and is in accordance with the results from Stone et al. [6] showing high levels of seric IL-6 produced in IRS+ patients. We show here that both phenomenon correlate with the abrupt onset of commonly reported clinical findings [2]: lymph node enlargement and functional granuloma formation [17]. We therefore propose a two step pathophysiology for IRS: the first event is an excessive restoration of PPD-specific Th1 response with no Th2 balance, responsible for enlargement of TB granuloma lesions, rapidly leading to an acute release of non-specific pro-inflammatory cytokines and chemokines inducing the systemic inflammatory syndrome. This latter findings support the use of systemic anti-inflammatory and/or immunosuppressive agents in severe IRS cases.

This mechanism can probably be extended to non-HIV patients experiencing equivalent paradoxical reactions during anti-TB therapy. In some non-HIV patients, IFN-γ secretion is missing during active TB infection, reappears during anti-mycobacterial treatment [18]. This desinhibition of IFN-γ production may be particularly strong concurrently to the restoration of immune response in profoundly immune-suppressed patients. No identified factors explain why this physiologic response to mycobacterial antigens becomes so intense and sometimes life-threatening in some patients, but genetic factors regulating Th1 functions may determine predisposition to IRS and its severity as already proposed by P. Price [19].

In conclusion, an acute exacerbation of Th1 responses against some mycobacterial antigens appears to cause IRS in patients co-infected with HIV and TB.

### EXAMPLE 2

### IRS is Associated with an Explosive Th1 Response Specifically Directed against Tuberculin (PPD) and the 16 KDa Protein

### Materials and Methods

### Subjects and Patients:

Fourteen HIV- and TB-coinfected patients (7 IRS+ and 7 IRS−) whom median CD4 cells count was 58/mm³ at baseline (time of TB diagnosis, T_{TB}) were tested for anti-mycobacterial responses at baseline and either at time (or after) of IRS for the IRS+ patients or between 1 to 6 months after the HAART onset for the IRS− patients. Four TB- and HIV-negative BCG-vaccinated and one non-vaccinated subjects were tested as controls. Four HIV-negative but TB-positive patients and one HIV-positive but TB-negative patients were tested also as control.

### Assays:

Antigen-specific Th1 cells producing IFN-γ were quantified on fresh and frozen peripheral-blood-mononuclear-cells (PBMC) using two series of assays:
ELISpot, as described (Martinez V, JID 2005), after a 40-hour stimulation with antigen controls included phytohaemagglutinin (Murex) and medium alone. Spots were counted using an ELISpot reader (Zeiss) and data were expressed as spot-forming-cells (SFC)/10⁶ PBMCs. Results were considered as positive if above a 50 SFC/10⁶ PBMC after subtraction of the mean background obtained with cells alone.
Intracellular Cytokine Staining (ICS) of IFN-γ-producing-cell was measured after 16-hour stimulation with or without PPD (10 μg/mL), native 16 kDa, secretion was blocked by brefeldine A, and staining was analyzed on a FACSCalibur.

### Antigens:

Mycobacterial extracts (PPD, 1 μg/ml; Statens-Serum-Institute, Copenhagen, Denmark), ESAT-6 recombinant protein (1 μg/ml, kindly provided by the Institute of Colorado State University, Colorado, USA). Native 16 kDa protein was kindly provided by the ICSU. The following antigens, kindly provided by the ICSU (USA) were also tested: recombinant Acr1 (HSPX), native 38 kDa, recombinant Ara- and Man-LAM (5, 20, 5, 20 and 20 μg/ml respectively). Acr2, TB10.4, TB10.3, Apa, recombinant PhosI (kindly provided by the Claude Leclerc, Institute Pasteur, Paris) were tested at optimized concentrations. The RD 1, CFP 10 antigen was tested in the commercial T-spot TB® test (Oxford Immunotech, Oxford, Chapman A, AIDS 2002) according to the manufacture recommendations.

Fourteen synthetic 20-mer overlapping neighboring peptides by 10 amino acids spanning the sequence of 16-kDa protein (Genepep®), with a purity of at least 80% and an HPLC confirmed homogeneity, were tested individually or pooled at optimized concentration.

### Results

### Identification of the Antigens Targeted in IRS

The antigens targeted in this tuberculin-specific response have been explored with ELISpot IFN-gamma (results are shown in table 2) in order to define precisely which were the antigens contained in the tuberculin preparation that were recognized in IRS and to prepare the basis for a solid diagnostic assay.

TABLE 2
ELISpot IFN-γ Antigen-specific T-cell quantification expressed as Spot Forming Cells (SFC)/10⁶ PBMC

| | | | TB− BCG | | | | HIV− TB+ | | HIV+ TB+ | | HIV+ BK+ | | |
|---|---|---|---|---|---|---|---|---|---|---|---|---|---|
| | TB− BCG | | Vaccinated | | | | patients | | T_{TB} | | IRS- | | |
| | Non-vaccinated | | n = 4 | | HIV+ TB− | | n = 4 | | n = 11 | | n = 7 | | |
| | n = 1 | | | SFC/10⁶ | n = 1 | | | SFC/10⁶ | | SFC/10⁶ | | SFC/10⁶ | |
| | | SFC/10⁶ | | PBMC | | SFC/10⁶ | | PBMC | | PBMC | | PBMC | |
| | N pos | PBMC | N pos | (median) | N pos | PBMC | N pos | (median) | N pos | (median) | N pos | (median) | p¹ |
| PPD | 0 | 43 | 4 | 303 | 0 | 0 | 4 | 420 | 7 | 80 | 6 | 425 | * |
| Rec ESAT6 | 0 | 26 | | 17 | 0 | 23 | 4 | 227 | | 12 | 1/5 | 10 | NS |
| CFP-10 | | ND | | ND | | ND | | ND | 0/1 | 2 | 2/3 | 100 | NS |
| native | 1 | 53 | 4 | 577 | 0 | 7 | 4 | 572 | 7 | 90 | 6 | 480 | * |
| 16 kDa | | | | | | | | | | | | | |
| (Acr1) | | | | | | | | | | | | | |
| Rec 16 KD | 0 | 13 | 3/3 | 357 | 0 | 10 | 2 | | 0 | 0 | 2 | 7 | NS |
| 16 kD | 0 | 0 | 2/3 | 440 | 0 | 0 | 1/2 | 28 | 5/9 | 0 | 1/3 | 0 | § |
| peptides | | | | | | | | | | | | | |
| Acr2 | 0 | 0 | 2/2 | 473 | 0 | 23 | 3 | 172 | 2 | 0 | 1 | 0 | NS |
| Native | 1 | 270 | 3/3 | 1200 | 0 | 10 | 2/2 | 123 | 3/10 | 30 | 3/4 | 155 | NS |
| 38 kDa | | | | | | | | | | | | | |
| Rec 38 kDa | 1 | 93 | 3/3 | 1673 | 0 | 17 | 2/2 | 220 | 5 | 43 | 2/4 | 87 | NS |
| (phosI) | | | | | | | | | | | | | |
| Apa | 0 | 0 | 1/2 | 43 | 0 | 13 | 1 | 43 | 4/10 | 35 | 1/4 | 9 | NS |
| TB 10.4 | 0 | 7 | 2/2 | 2020 | 0 | 17 | 4 | 432 | 9/10 | 103 | 5 | 127 | NS |
| TB 10.3 | 0 | 0 | 2/2 | 1050 | 0 | 0 | 3 | 205 | 5/10 | 40 | 4 | 73 | NS |
| Ara LAM | | ND | 0/2 | 13 | | ND | 0/2 | 0 | 0/1 | 0 | 2 | 30 | NS |
| Man LAM | 0 | 0 | 1/2 | 100 | 0 | 0 | 2 | 48 | 3 | 7 | 2 | 27 | NS |

ND = not done,
N pos = number of positive results/tested,
T_{TB} = anti mycobacterain therapy onset,
¹Non parametric Mann Whitney test between IRS+ and IRS− data (SFC/10⁶ PBMC).
* p < 0.05,
§ p = 0.07

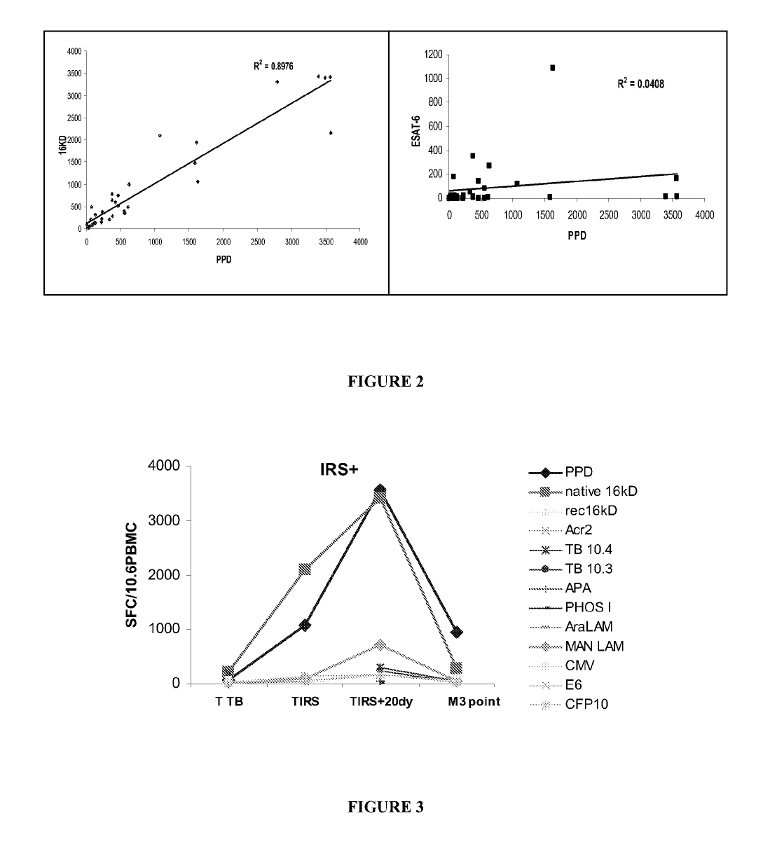

First of all, we found a high correlation (FIG. 2) between PPD and native 16 kDa-specific responses quantified with ELISpot in all subjects tested, even in highly immunocompromised subjects as HIV-infected patients. This high correlation was not found with the other antigens.

At time of IRS, as observed for PPD, the 16 kDa-specific response claimed sharply in the IRS+ patient tested (FIG. 3) and was significantly higher than in the IRS− patients (table 2). This was not true for the others antigens tested specially the RD1-antigens (ESAT-6 and CFP-10).

### Phenotypic Characterization of Tuberculin- and 16 KDa-Specific T-Cells in IRS

At time of IRS, 14% of the PPD- and 16 kDa-specific cells are CD8+, 80% are CD4+, and represents up to 35% of the total CD4. These CD4 T-cells are activated (100% are HLA-DR+), co-produce TNF-α but not IL-2 and are effector memory cells (84% are CD45RA-CD27−).

### Conclusion

Our results demonstrate that IRS is associated with both PPD- and a native 16 kDa-specific Th1-cell responses but not with T cells responses directed against other TB antigens. These findings suggest IRS is due to an explosive reactivation of the effector memory CD4 T cells directed against TB antigens from the dormancy stage but not against secreted antigens or other antigens associated with active tuberculosis. The responses can be induced by native protein as well as immunodominant peptides of 16 kDa. Therefore these results provide unique tools for the immunological diagnosis of IRS, allowing to distinguish between a TB relapse and IRS.

### REFERENCES

1. French M A, Mallali S A, Dawkins R L. Zidovudine-induced restoration of cell-mediated immunity to mycobacteria in immunodeficient HIV-infected patients. Aids 1992; 6:1293-1297.
2. Lawn S D, Bekker L G, Miller R F. Immune reconstitution disease associated with mycobacterial infections in HIV-infected individuals receiving antiretrovirals. Lancet Infect Dis 2005; 5:361-373.
3. French M A, Price P, Stone S F. Immune restoration disease after antiretroviral therapy. Aids 2004; 18:1615-1627.
4. Dean G L, Edwards S G, Ives N J, Matthews G, Fox E F, Navaratne L, et al. Treatment of tuberculosis in HIV-infected persons in the era of highly active antiretroviral therapy. Aids 2002; 16:75-83.
5. Morlese J F, Orkin C M, Abbas R, Burton C, Qazi N A, Nelson M R, et al. Plasma IL-6 as a marker of mycobacterial immune restoration disease in HIV-1 infection. Aids 2003; 17:1411-1413.
6. Stone S F, Price P, Keane N M, Murray R J, French M A. Levels of IL-6 and soluble IL-6 receptor are increased in HIV patients with a history of immune restoration disease after HAART. HIV Med 2002; 3:21-27.
7. Li T S, Tubianai R, Katlama C, Calvez V, Ait Mohand H, Autran B. Long-lasting recovery in CD4 T-cell function and viral-load reduction after highly active antiretroviral therapy in advanced HIV-1 disease. Lancet 1998; 351:1682-1686.
8. Autran B, Carcelain G, Li T S, Blanc C, Mathez D, Tubiana R, et al. Positive effects of combined antiretroviral therapy on CD4+ T cell homeostasis and function in advanced HIV disease. Science 1997; 277:112-116.
9. Narita M, Ashkin D, Hollender E S, Pitchenik A E. Paradoxical worsening of tuberculosis following antiretroviral therapy in patients with AIDS. Am J Respir Crit Care Med 1998; 158:157-161.
10. Martinez V, Costagliola D, Bonduelle O, N'go N, Schnuriger A, Theodorou I, et al. Combination of HIV-1-specific CD4 Th1 cell responses and IgG2 antibodies is the best predictor for persistence of long-term nonprogression. J Infect Dis 2005; 191:2053-2063.
11. Carson R T, Vignali D A. Simultaneous quantitation of 15 cytokines using a multiplexed flow cytometric assay. J Immunol Methods 1999; 227:41-52.
12. Havlir D V, Schrier R D, Torriani F J, Chervenak K, Hwang J Y, Boom W H. Effect of potent antiretroviral therapy on immune responses to Mycobacterium avium in human immunodeficiency virus-infected subjects. J Infect Dis 2000; 182:1658-1663.
13. Wendland T, Furrer H, Vernazza P L, Frutig K, Christen A, Matter L, et al. HAART in HIV-infected patients: restoration of antigen-specific CD4 T-cell responses in vitro is correlated with CD4 memory T-cell reconstitution, whereas improvement in delayed type hypersensitivity is related to a decrease in viraemia. Aids 1999; 13:1857-1862.
14. Hengel R L, Allende M C, Dewar R L, Metcalf J A, Mican J M, Lane H C. Increasing CD4+ T cells specific for tuberculosis correlate with improved clinical immunity after highly active antiretroviral therapy. AIDS Res Hum Retroviruses 2002; 18:969-975.
15. Foudraine N A, Hovenkamp E, Notermans D W, Meenhorst P L, Klein M R, Lange J M, et al. Immunopathology as a result of highly active antiretroviral therapy in HIV-1-infected patients. Aids 1999; 13:177-184.
16. Mori T, Sakatani M, Yamagishi F, Takashima T, Kawabe Y, Nagao K, et al. Specific detection of tuberculosis infection: an interferon-gamma-based assay using new antigens. Am J Respir Crit Care Med 2004; 170:59-64.
17. Lawn S D, Macallan D C. Hypercalcemia: a manifestation of immune reconstitution complicating tuberculosis in an HIV-infected person. Clin Infect Dis 2004; 38:154-155.
18. Hirsch C S, Toossi Z, Othieno C, Johnson J L, Schwander S K, Robertson S, et al. Depressed T-cell interferon-gamma responses in pulmonary tuberculosis: analysis of underlying mechanisms and modulation with therapy. J Infect Dis 1999; 180:2069-2073.
19. Price P, Morahan G, Huang D, Stone E, Cheong K Y, Castley A, et al. Polymorphisms in cytokine genes define subpopulations of HIV-1 patients who experienced immune restoration diseases. Aids 2002; 16:2043-2047.

1. A method of in vitro diagnosis of Immune Restoration Syndrome (IRS) in patients co-infected with mycobacterium tuberculosis (TB) as well as in patients infected with TB alone, comprising the step consisting of:
a) incubating peripheral blood mononuclear cells (PBMC) or whole blood of said patients in a culture medium with mycobacterial antigens selected from mycobacterial extract referred as PPD (purified protein derivative), and/or with the 16 kDa protein (SEQ ID No. 1);
b) incubating peripheral blood mononuclear cells (PBMC) or whole blood of said patients in a culture medium with at least one antigen from mycobacterium tuberculosis selected from ESAT-6 (SEQ ID No. 2), CFP-10 (SEQ ID No. 3) and 85B (SEQ ID No. 4) as negative control; and
c) detecting the level of the Th1 response against said mycobacterial antigens described in a) compared to said mycobacterial antigens in b), wherein an increase above 250% of Th1 response, or a strong reactivity in the Th1 response measured at one time point against one of the antigens as defined in a) compared to the antigens as defined in b) or compared to non-stimulated cells or control cells stimulated with non-mycobacterial antigens is indicative of IRS.

2. The method according to claim 1, wherein step b) comprises performing a quantification of antigen-specific T-cells by ELISPOT assay or intra-cellular cytokine staining combined to flow cytometry or fluorescent microscopic examination and quantification.

3. The method according to claim 2, wherein it comprises quantification of mycobacterial-specific Th1 cells producing IFN-γ.

4. The method according to claim 2 or 3, wherein a result above 500 SFC (spot-forming-cells)/10⁶ PBMC, for example a SFC above 500, 1000, or 2000, after subtraction of the mean background obtained with cells alone, is an indication of IRS; while simultaneously no or minimal SFC are detected against ESAT-6, 85B, CFP-10, such as SFC typically below 500/10⁶ PBMC or below 400 or 300.

5. The method according to one of claims 1 to 4, wherein step b) comprises collecting culture supernatants and quantifying at least one Th1 specific cytokine or chemokine, including IL-2, IL-2R, IL-12p40, IL-15, IL-17, IFN-γ, TNF-α, GMCSF, MIP-1α, MIP-1β, MCP-1, MIG, RANTES, IP-10.

6. The method according to claim 5 which is performed using a multiplexed sandwich immunoassay or an individual cytokine-specific sandwich immuno-assay.

7. The method according to claim 5, wherein IFN-γ production is quantified by ELISA.

8. The method according to one of claims 1 to 7, wherein peripheral blood mononuclear cells in step a) are incubated from about 6 to 72 hours.

9. The method according to one of claims 1 to 8, wherein peripheral blood mononuclear cells in step a) are incubated with PPD and/or the 16 kDa protein at about 1 μg/ml and separately with ESAT-6 recombinant protein at about 1 μg/ml.

10. The method according to one of claims 1 to 9, wherein peripheral blood mononuclear cells in step a) are incubated with PPD and/or the 16 kDa protein at about 1 μg/ml and separately with CFP-10 and/or 85B.

11. The method according to one of claims 1 to 10, wherein control cells are selected from cells incubated with CMV extracts, HIV-1 p24, phytohaemagglutinin and medium alone.

12. The method according to one of claims 1 to 11, which is performed in the course of Highly Active Antiretroviral Therapy (HAART) in HIV positive patients co-infected with tuberculosis.

13. The method according to one of claims 1 to 12, which is performed in patients receiving simultaneous or sequential HAART and tuberculosis treatment.

14. The method according to one of claims 1 to 11, which is performed in patients with TB alone.

15. A diagnostic kit of Immune Restoration Syndrome (IRS) in TB patients or patients co-infected with tuberculosis (TB) and HIV, comprising mycobacterial antigens selected from mycobacterial extract referred as PPD (purified protein derivative) and the 16 kDa protein (SEQ ID No. 1), and at least one antigen from mycobacterium tuberculosis selected from ESAT-6 (SEQ ID No. 2), CFP-10 (SEQ ID No. 2) and 85B (SEQ ID No. 3).

16. The diagnostic kit according to claim 15 further comprising at least one anti-cytokine/chemokine antibody specific to the Th1 immune response bound to a solid support, such as anti-IFN-γ antibodies.

17. The diagnostic kit according to claim 16 wherein the solid support is an ELISPOT plate.

18. The diagnostic kit according to claim 16 wherein the solid support is an ELISA plate.

19. The use of the kit according to one of claims 15 to 18 for detecting tuberculosis related Immune Restoration Syndrome (TB-IRS) in TB patients or patients co-infected with tuberculosis (TB) and HIV in course of HAART and tuberculosis treatment.

20. The use of the kit according to one of claims 15 to 18 for detecting Immune Restoration Syndrome (TB-IRS) in patients with TB alone.
